(19) United States
(12) Patent Application Publication
(10) Pub. No.: US 2011/0306985 A1
(43) Pub. Date: Dec. 15, 2011

(54) Surgical Assistance System

(75) Inventors: Takayuki INOUE, Okayama-shi (JP); Koichi KURAMOTO, Okayama-shi (JP); Yoshio NAKASHIMA, Okayama-shi (JP); Naohiko SUGITA, Tokyo (JP); Mamoru MITSUISHI, Tokyo (JP); Yoshikazu NAKASHIMA, Tokyo (JP)

(73) Assignees: National University Corporation Tokyo University; Nakashima Medical Co., Ltd., Okayama-shi (JP)

(21) Appl. No.: 12/986,848

(22) Filed: Jan. 7, 2011

(30) Foreign Application Priority Data
Jun. 9, 2010 (JP) 2010-131646

(51) Int. Cl. A61B 19/00

(52) U.S. Cl. 606/130

(57) ABSTRACT

A surgical assistance system for operating on biological tissue using a surgical tool attached to an arm of an automatically-controlled surgical instrument so that an optimal feed rate of the tool is calculated and outputted to the surgical instrument, the system including: a device for storing and voxelizing medical image data obtained from a biological tissue subject to surgery; a device for setting an operative location based on the shape of the biological tissue; a device for calculating a tool path along which the tool travels to perform surgery at an operative location; a device for determining the region of interference between the tool and the voxels; a device for determining the hardness of the biological tissue in the interference region; a device for calculating an optimal tool feed rate corresponding to the hardness; and a device for outputting the feed rate obtained by the calculations to the surgical instrument.

### BACKGROUND OF THE INVENTION

1. Field of the Invention

The present invention relates to a surgical assistance system that optimizes the tool feed rate in order to ensure minimal invasiveness and save time during surgery when operating on biological tissues during artificial joint replacement, etc. with the help of a surgical instrument based on a computerized robotic system.

2. Description of the Related Art

In recent years, surgical instruments based on computerized robotic systems, in other words, surgical robots, have been used for the purpose of ensuring reduction in burden and early recovery of patients by using surgery that produces small wounds (by minimally invasive surgery) and increasing the accuracy of surgery with a view to improve in-vivo lifetime by reducing the load acting on the biological tissues and implant components, such as artificial joints and the like. For example, in order to perform accurate surgery on a bone (hereinafter referred to as “resection”) for artificial joint implantation during artificial joint replacement surgery, such an arrangement has been used that a surgical tool (hereinafter referred to as “the tool”) is attached to an arm of a surgical instrument, such as the one disclosed in Patent Document 1 below, and the target bone is resected by automatically controlling this tool so as to conform to the shape of an artificial joint-receiving surface.

Just as in the case of machine parts and other work pieces processed by regular machine tools, maintaining the relative positional relationship between the tool and the work piece is important in ensuring accurate resection of biological tissues in accordance with a pre-operative plan. Since ordinary metallic materials possess a high rigidity, they can be firmly secured (clamped) to the table of a machine tool. Biological tissues, however, have extremely low rigidity in comparison with metallic materials and are therefore difficult to secure in a rigid manner. For example, in the case of bone tissue, its rigidity is higher than that of the skin or muscle tissues, but securing a bone by retaining it directly with fixtures is possible only in a limited range of circumstances because, outside of the surgical site, the periphery of the bone is usually surrounded by soft tissue.

In addition, while minimally invasive surgery has been used recently for the purpose of ensuring physical reduction in burden and early post-operative recovery of patients, such surgery offers even fewer options for directly retaining affected bones. For this reason, despite the fact that fixtures used for securing bones through the medium of soft tissue, such as the one shown in Patent Document 2 below, have been proposed in the past, the use of such fixtures does not provide a retaining force as strong as the one used to hold work pieces in place during machining.

Furthermore, while reducing operating time is important in alleviating the physical burden on the patients, doing so in practice implies increasing the feed rate of a tool, including the incision depth of the tool, for the purpose of reducing the time of bone resection. However, if the feed rate is raised, the cutting load will also be increased, and therefore the retaining force that needs to be applied to the affected site to maintain the relative positional relationship between the tool and the biological tissue will have to be made even stronger. However, as described above, there is a limit to the retaining force applied to an affected site in biological tissue. Thus, meeting the requirements for accurate resection of biological tissue, minimal invasiveness, and reduced operating room time requires resection with the proviso that the cutting load is set to or below a predetermined value.

In particular, due to the fact that the hard-surfaced cortical bone and inner cancellous bone exhibit a gradient structure in the bone tissue, there is significant variation in the cutting load during resection. This induces vibration in the tool-gripping arm of a surgical instrument and the affected site and produces biological tissue displacement at those locations where the cutting load is high. Methods used to minimize the vibration of arm and displacement of the biological tissue include a method that measures cutting reaction force that acts on a tool during resection by way of using a force sensor so as to control the feed rate via force feedback in real time. However, if the feed rate control is exercised subsequent to force detection, it is difficult to exercise precise control in a biological tissue resection system, in which the retaining force is hard to ensure. This causes the biological tissue to be displaced under the action of the cutting load and brings about a decrease in surgical accuracy.

### PATENT DOCUMENTS

[Patent Document 1] Japanese Patent Application Laid-Open (Kokai) No. 2002-306500

[Patent Document 2] Japanese Patent Application Laid-Open (Kokai) No. 2007-202950

[Patent Document 3] Japanese Patent Application Laid-Open (Kokai) No, 2008-146457

### BRIEF SUMMARY OF THE INVENTION

Accordingly, the present invention, taking into consideration of various characteristics of biological tissue in a resection region, including its gradient material characteristics, provides accurate resection of biological tissue, minimal invasiveness, and reduced operating time by setting cutting load to or below a specified value while ensuring a feed rate that is optimal in each case.

In order to attain the above-described objects, the present invention provides a surgical assistance system for operating on biological tissue using a surgical tool attached to an arm of an automatically-controlled surgical instrument, and this system is characterized in that it comprises: a means for storing and voxelizing medical image data obtained from a biological tissue subject to surgery; a means for setting an operative location based on the shape of the biological tissue; a means for calculating a tool path traveled by the surgical tool to perform surgery at the operative location; a means for determining the region of interference between the surgical tool and voxels; a means for determining the hardness of the biological tissue in the interference region; a means for calculating an optimal tool feed rate corresponding to the determined hardness; and a means for outputting the feed rate obtained by the calculations to the surgical instrument.

In addition, in the above-described surgical assistance system, the present invention provides a means for estimating the hardness of the biological tissue based on the brightness values of the medical images and calculating a feed rate based on the estimated hardness, and further provides a means for measuring the cutting reaction force acting on the tool in real time and calculating a feed rate corresponding to the cutting reaction force using force feedback.

According to the invention, control over the feed rate is exercised by predicting the hardness of the biological tissue in advance such that cutting loads higher than a predetermined value do not act on the biological tissue, thus reducing vibration and biological tissue displacement during resection. As a result, resection can be performed at the optimal feed rate, which allows for accurate resection to be performed and makes it possible to reduce the time and invasiveness of the surgery. According to the invention, bone hardness can be estimated using a simple method, and it is possible to exercise feed rate control based on additional force feedback information, minimizes excessive increases in the cutting load due to biological tissue displacement along the tool path or in real space, and ensures accurate resection of the biological tissue.

### BRIEF DESCRIPTION OF THE SEVERAL VIEWS OF THE DRAWINGS

FIG. 1 is an explanatory diagram of the surgical assistance system according to the preset invention.

FIG. 2 is a block diagram of the hardware used in the surgical assistance system.

FIG. 3 is a schematic diagram illustrating the concept of registration.

FIG. 4 is a schematic diagram illustrating the concept of tool element segmenting and interference determination.

FIG. 5 is a diagram illustrating an exemplary tool path and tool orientation information.

FIG. 6 is an explanatory diagram illustrating the extraction of the finite elements of the tool that correspond to the resection region.

FIG. 7 is an explanatory diagram illustrating the NC codes used to execute the surgical assistance system.

FIG. 8 is an enlarged reference schematic view of the left side of FIG. 4, specifying the entered numerical values.

FIG. 9 is an enlarged reference schematic view of the right side of FIG. 4, specifying the entered numerical values.

### DETAILED DESCRIPTION OF THE INVENTION

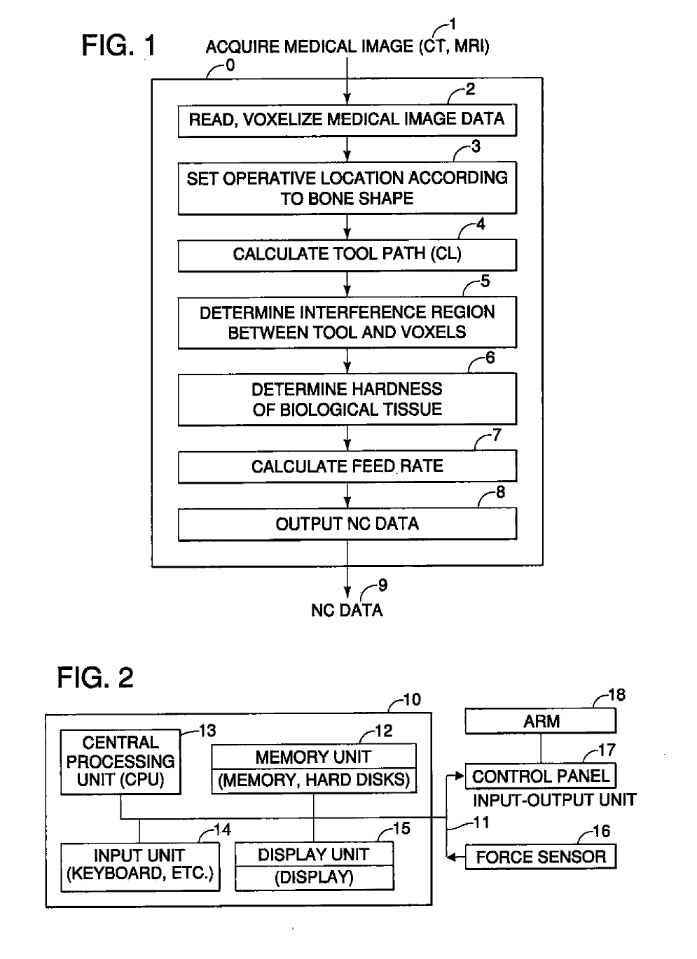

A description of an embodiment of the present invention will be given below with reference to examples illustrating the resection of tibia and femur, i.e. the bones used for artificial knee joint replacement, as the biological tissue at the operative location. FIG. 1 is a block diagram illustrating the various means that constitute the present invention. The present invention comprises: a means 1 for acquiring medical images from a biological tissue in the area around the knee (referred to as “the bone” below), a means 2 for storing and voxelizing the acquired medical images, a means 3 for setting an operative location depending on the shape of the bone, a means 4 for calculating a tool path along which the tool to perform resection travels at the operative location, a means 5 for determining the region of interference between the tool and the voxels, a means 6 for determining the hardness of the biological tissue in the interference region, a means 7 for calculating the optimal tool feed rate corresponding to the hardness, and a means 8 for outputting the feed rate obtained by the calculations to the surgical instrument as NC data 9. With these means, a surgical assistance system (referred to as “the assistance system” below) 10 is provided.

The main portion of the assistance system 10 is a computer, and it is comprised of: an input-output unit 11 (generally a CD-ROM or DVD drive, or a LAN, etc., with the LAN used for real-time output), which is for acquiring information from outside and transmitting information outside; a memory unit 12 (a ROM (Read-Only Memory), a RAM (Random Access Memory), and a hard disk, on which an OS or an OS-like program is stored) which stores information obtained from outside and computational information internally executed as well as computational programs; a central processing unit (Central Processing Unit) 13 which performs calculations using the internal computational information and programs; an input unit 14 (a device such as a keyboard, a mouse, etc.) used to set specified values, etc.; and a display unit 15 (a display) that displays information.

Information storage takes place in the memory unit 12 (in some cases, when the volume of information stored in the ROM or RAM is extremely large, some of the information is stored on the hard disk), calculations are performed in the central processing unit 13 with the help of the programs stored in the memory unit 12, the input of various instructions and cutting load settings (specified values) depending on the hardness etc. of the bone is performed via the input unit 14, and the acquisition of information from outside, as well as the transmission of information from the system, is performed via the input-output unit 11. Each one of the above-described means will be described below in greater detail.

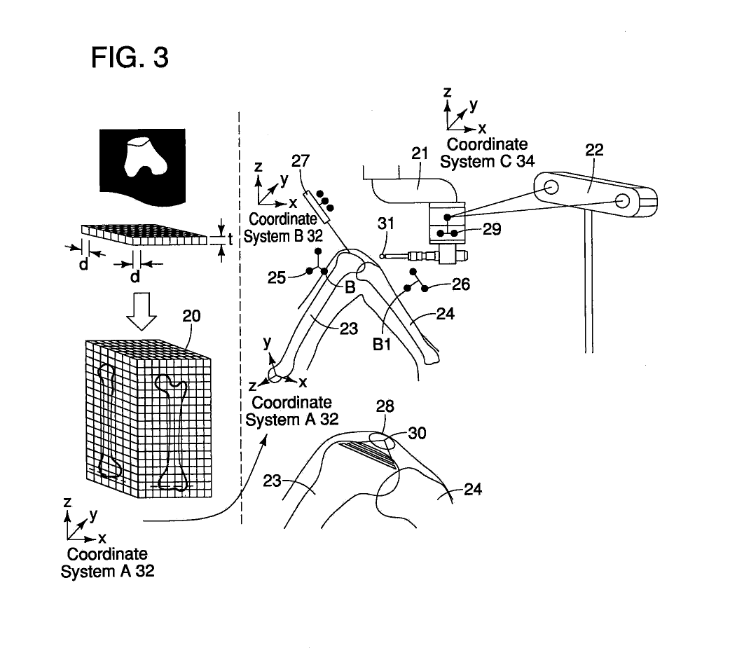

First, a description will be given for the means 2 for storing and voxelizing medical image data relating to the bone that undergoes surgery. In the shown example, DICOM (Digital Imaging and Communication in Medicine)-formatted multi-slice CT (Computer Tomography) data is used as the above-described medical images. The DICOM data stores image thickness data, as well as image slice positions and pixel sizes. The three-dimensional voxel data, which is based upon the pixel size d and image thickness t internally defined in the DICOM data, is created as shown in FIG. 3. The location of the centers and image brightness values (CT values, to be described below, are used in the shown example) of all the voxel elements in the voxel data are stored in the memory unit 12.

Depending on the relationship between the thickness of the voxels and the inter-slice spacing of the tomographic images obtained by CT imaging, gaps may appear between the slices. In such cases, the voxel elements of the gap portion are interpolated. Such interpolation may involve linear interpolation, which allows for realistic voxel data to be obtained by inserting adjacent voxel elements between the slices in a linear fashion. It should be noted that inter-slice interpolation may use non-linear interpolation methods based on various principles. Conversely, if there is overlapping between the tomographic images, adjacent voxel elements in the overlapping portion are attenuated. Furthermore, although the shown example uses CT images, MRI (Magnetic Resonance Imaging system) tomographic image data may be used as well. The image capture area of the medical images should be sufficiently large to include the entire region of resection performed by the tool.

The means 3 for setting the resection surface in accordance with the shape of the bone will described below. First of all, it acquires information on the shape of the bone, i.e. the operative location to be resected. Since the objective in the shown example is artificial knee joint replacement, the shapes of the bones (the femur and the tibia) are extracted from the voxel data based on the CT images, and the location, where the artificial joint is to be placed, is determined with reference to the shape data. In the shown example, the brightness values of the voxel data are used to recognize voxel data elements located within a specific brightness value range as bone tissue and the shape of the bone surface is built using the Marching Cubes algorithm. The implantation position of the artificial joint is determined with reference to the shape of the bone surface, thereby making it possible to determine and digitize the position of the artificial joint-receiving surface (i.e., the location of bone resection) relative to the bone surface shape data.

The above-described bone surface shape is built in the central processing unit 13 based on the voxel data stored in the memory unit 12 and is displayed on the display unit 15 as a shape of the bone. In addition, data concerning the shape of the artificial joint is also stored in the memory unit 12, and the shape and position of the artificial joint are displayed on the display unit 15 together with the shape of the bone surface. The position of the artificial joint is adjusted depending on the values inputted via the input unit 14. The position of the artificial joint is determined according to a doctor's judgment by comparing the artificial joint shape and the shape of the bone surface displayed on the display unit 15.

Here, the above-described bone surface shape can be built and the position etc. of the artificial joint can be specified using a DICOM viewer (for example, a program called “Mimics” available from Materialise) etc. on a general-purpose computer. In such a case, the bone surface shape data and the digitized resection location information, etc., are transferred from the input-output unit 11 and stored in the memory unit 12. In addition, while STL (Stereo Lithography) is a suitable format for the acquired bone surface shape data and artificial joint shape data, information in the form of general-purpose CAD data based on TOES, etc., may be used as well.

In the means 4 for calculating a tool path along which the tool travels for shaping the artificial joint-receiving surface, the tool path (CL-Cutter Location) is calculated based on resection surface information 20 determined from the artificial joint implantation position and bone surface shape data. Furthermore, the orientation of the tool is calculated in addition to the tool path (this information is collectively referred to as “tool information 30”). When the tool path and tool orientation are calculated, as a first step, a voxel coordinate system 32, which is used to represent voxel data built from multi-slice CT data as shown in FIG. 3 (since the shape of the bone surface is built from this voxel data, it is represented in the same coordinate system together with the position of the artificial joint-receiving surface (resection surface)), is translated to a surgical instrument coordinate system 34 used by the arm 21 of the surgical instrument, which has a tool 31 attached to its distal end and is used to perform the actual resection. In the shown example, the coordinate system translation is performed using an infrared coordinate measurement machine (trade name “Polaris” manufactured by Northern Digital, Inc.). The translation procedure is outlined below.

Referring to FIG. 3, first, the voxel coordinate system 32, which is used to represent the resection surface information 20, is translated to a real space coordinate system 33 used to represent the bones (the femur 23 and the tibia 24) in the space of the operating room. In this case, trackers for infrared measurement (25 for the femur, 26 for the tibia) are attached to the bones 23, 24 and are used as a reference for setting up the real space coordinate system 33. The probe 27 of the infrared coordinate measurement machine is used to measure the shape of the bones 23, 24 through a percutaneous incision 28 relative to the real space coordinate system 33, and registration is performed in order to minimize any discrepancies between the information on the shape of the bones 23, 24 obtained by measurement and the shape of the bone surface information acquired by the means 3 for setting the resection surface based on the shape of the above-described bones. As a result, the voxel coordinate system 32, which is used to represent the resection surface information 20, is associated (registered) with the real space coordinate system 33 used to represent the bones 23, 24 in the operating room.

This operation, which is performed in order to align the position of the actual bones 23 and 24 and the resection surface information 20, is referred to as “registration.” For example, such registration may be carried out using the method described in K. Saitou, et al., “Optimization of landmarks in point-based registration using point measurement error distribution estimation based on bone surface shape,” Journal of Japan Society of Computer Aided Surgery, vol. 10, no. 3, pp. 313-314 (2008), i.e. the above-described Patent Document 3. As a result of such registration, the voxel coordinate system 32 is translated to the real space coordinate system 33.

Next, the real space coordinate system 33 is translated to the surgical instrument coordinate system 34 based on a relative positional relationship determined by simultaneous measurement of the position of the tracker 29 attached to the arm 21 and the trackers 25, 26 attached to the bones 23, 24. Finally, the resection surface information 20, which represents the resection surface and the shape of the bones, is translated from coordinate values in the voxel coordinate system 32 to coordinate values in the surgical instrument coordinate system 34 of the arm 21 in accordance with the following formula (1).

P^{c}=T_{B}^{C} T_{A}^{B} P_{A} (1)

In this case,
P^{C}
is a coordinate value in the surgical instrument coordinate system 34,
P_{A}
is a coordinate value in the voxel coordinate system 32,
T_{B}^{C}
stands for information on translation (information on registration) from the real space coordinate system 33 to the surgical instrument coordinate system 34, and

### T_{A}^{B}

stands for information on registration between the voxel coordinate system 32 and the real space coordinate system 33.

Thus, translation to the coordinate systems 32, 33, and 34 is performed because the orientations of the respective coordinate axes are different and the angles and positions of the bones 23, 24 may be changed in order to avoid interference of the tool 31 and the probe 27 with the periosteal tissues during bone resection and bone shape measurement by the probe 27 through the percutaneous incision 28. In other words, once the coordinate translation from the voxel coordinate system 32 to the real space coordinate system 33 is finished, if there is a change in the position or orientation of the bones 23, 24, coordinate translation from the voxel coordinate system 32 to the surgical instrument 34 can be accomplished simply by performing a translation from the real space coordinate system 33 to the surgical instrument coordinate system 34, which eliminates the need to repeat the registration operation and reduces user effort.

As described above, a tool path and a tool orientation are calculated based on the resection surface information 20 for the bones 23, 24 translated to the surgical instrument coordinate system 34. This information is referred to as “tool information 30.” Methods such as the one described in the document “N. Sugita et al., ‘Bone cutting robot with soft tissue collision avoidance capability by a redundant axis for minimally invasive orthopedic surgery,’ Proceedings of IEEE/CME International Conference on Complex Medical Engineering (CME 2007)” are contemplated for use in calculating the tool information 30.

A description of the method set forth in the above-described document, as applied to the calculation of the tool information 30, is given below. First, the shape of the percutaneous incision 28 is measured in the surgical instrument coordinate system 34. This is done in order to avoid damage to the periosteal tissue as a result of interference of the tool 31 with the percutaneous incision 28 and the periosteal tissue and form the resection surface for the implantation of the artificial joint using a small wound (minimal invasiveness).

Next, the resection region of the bones 23, 24 is calculated using the bone surface shape data and the resection surface position contained in the resection surface information 20 translated to the coordinate system 34. The resection region of the bones 23, 24 is obtained by trimming (cutting away) and segmenting the bone surface shape data and using the segmented bone surface shape data of the portion that is cut away by the tool 31 as the resection region of the bones 23, 24. In the present embodiment, this operation is performed on the bone surface shape data of the femur 23 and tibia 24 translated to the surgical instrument coordinate system 34, and the resection region of the femur 23 and tibia 24 is determined.

After determining the resection region, a tool path is calculated based on a prescribed tool incision depth and overlap percentage such that the tool can cover the entire resection region. Furthermore, a tool orientation that prevents interference with the percutaneous incision 28 is determined for the calculated tool path based on the shape of the calculated percutaneous incision 28.

In the shown example, the arm 18 has thee (3) axes of translation and three (3) axes of rotation, which makes it possible to define any tool position or orientation. In the meantime, the tool information 30 is stated relative to the surgical instrument coordinate system 34 in the shown example. Such a method of calculating the tool information 30 permits bone resection for artificial joint implantation while avoiding damage to the periosteal tissue.

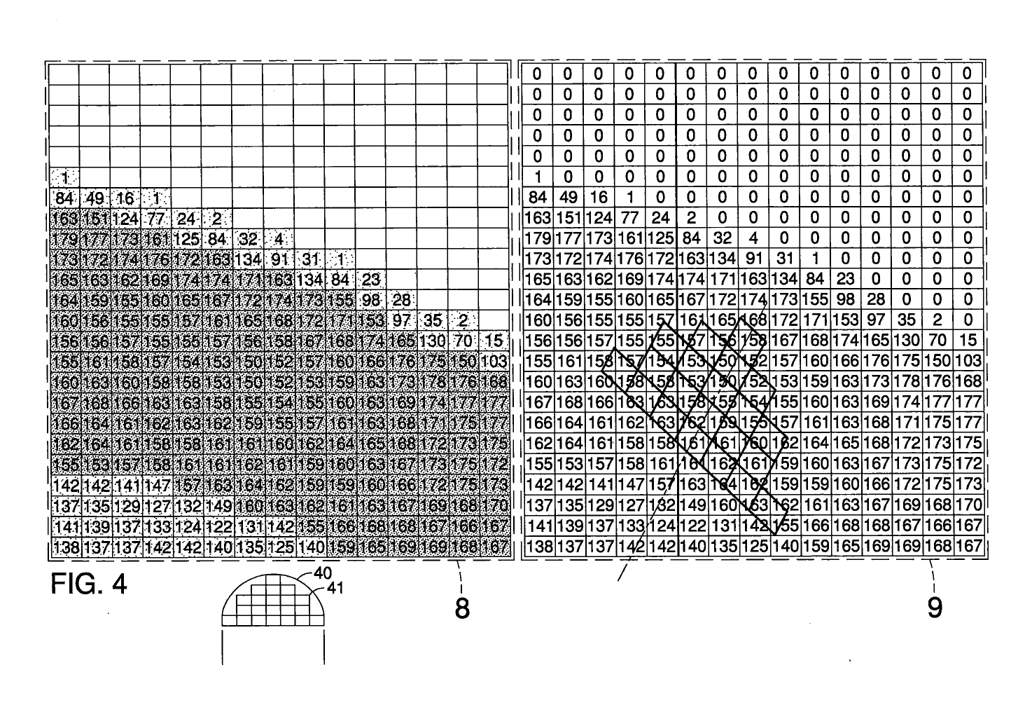

The means 5 for determining the region of interference between the tool 31 and the voxels is executed next. In this case, to optimize the feed rate, the first step is to calculate the hardness of the bones 23, 24. What is determined in such a case is the position of the tool, which is calculated based on information derived from the tool information 30, and the interference of the tool 31 with voxels generated from multi-slice CT data or other image information in this tool position, i.e. the corresponding location of the resection region in the voxels. As seen from FIG. 4, in the shown example, an approximate shape obtained by segmenting the shape of the cutting edge area 40 of the tool into finite elements 41 is used to determine whether there is interference between the tool 31 and the voxels. The procedure is described below.

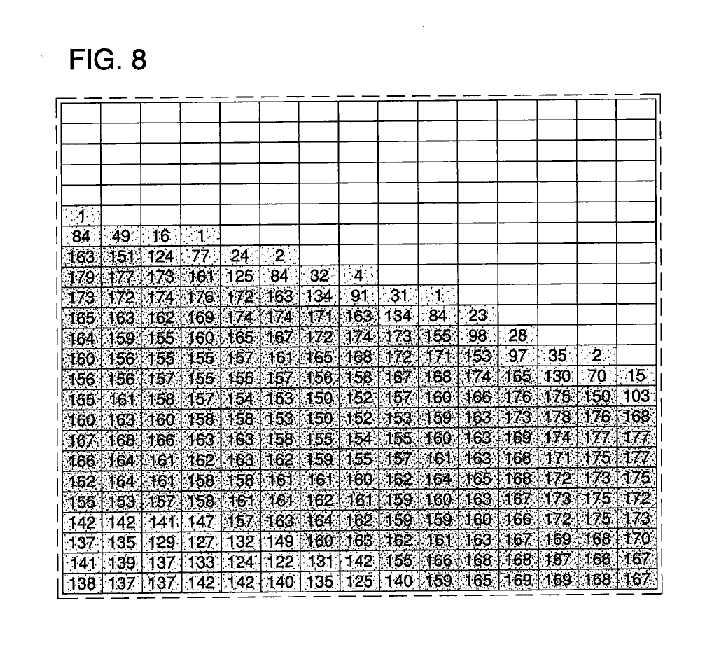

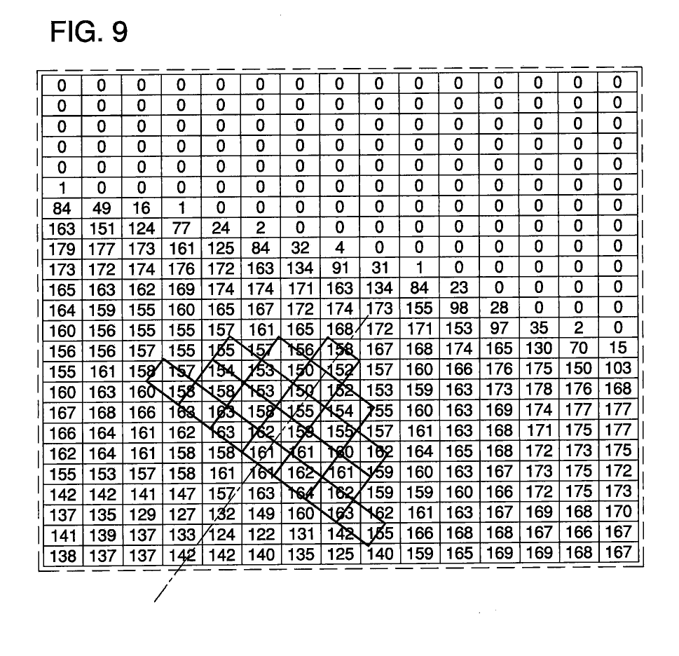

Incidentally, FIG. 9 or the right side chart in FIG. 4 is a slice image of arbitrary face of voxels, and the numbers inside correspond to the X-ray permeation rate in which the higher values mean the higher X-ray permeation rate (corresponding to the hardness of bones). FIG. 8 or the left side of chart in FIG. 4 is a chart in which the numbers that are the same as those in FIG. 9 are sorted by different shadings as generally seen in CT processing.

In the shown example, a ball nose end mill is used as the tool 31. The cutting edge region 40 of the ball nose end mill is segmented into finite elements 41 and is translated into the positions and orientations of finite elements 41 of the arm 21 in the surgical instrument coordinate system 34 based on information derived from the tool information 30. Furthermore, based on an inverse transform of formula (1) above, the finite elements 41 are translated from the surgical instrument coordinate system 34, which is used to represent the position of the arm 21 in FIG. 3, to the voxel coordinate system 32, which is used to represent voxels.

A determination as to the presence of interference is then made by superimposing and comparing the finite elements 41 of the region of the tool involved in resection, translated to the voxel coordinate system 32, and voxels formed from medical images, such as multi-slice CT data. Interference can be readily determined because the finite elements 41 of the tool 31 and voxels are represented in the same voxel coordinate system 32. In the shown example, the presence of interference is determined using the AABB (Axis-Aligned Bounding Box) method. However, it can be determined using other methods, such as the OBB (Oriented Bounding Box) method, and the like.

In other words, the voxel elements determined to be in interference with the finite elements 41 of the region of the tool that is involved in resection represent the region where the bone is cut by the tool in the tool position information Nᵢⱼ.

Here, voxels represented in the voxel coordinate system 32 are translated to the real space coordinate system 34, which is used to represent the position of the arm 21, thereby making it possible to perform interference determination by comparing them with the tool information 30 when the above-described tool information 30 is calculated. However, since the number of the finite elements 41 of the tool 31 is substantially smaller than the number of the voxel elements, the former method was adopted in order to ensure better calculation efficiency.

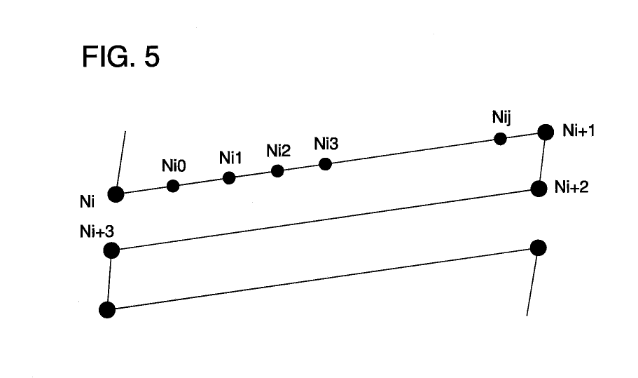

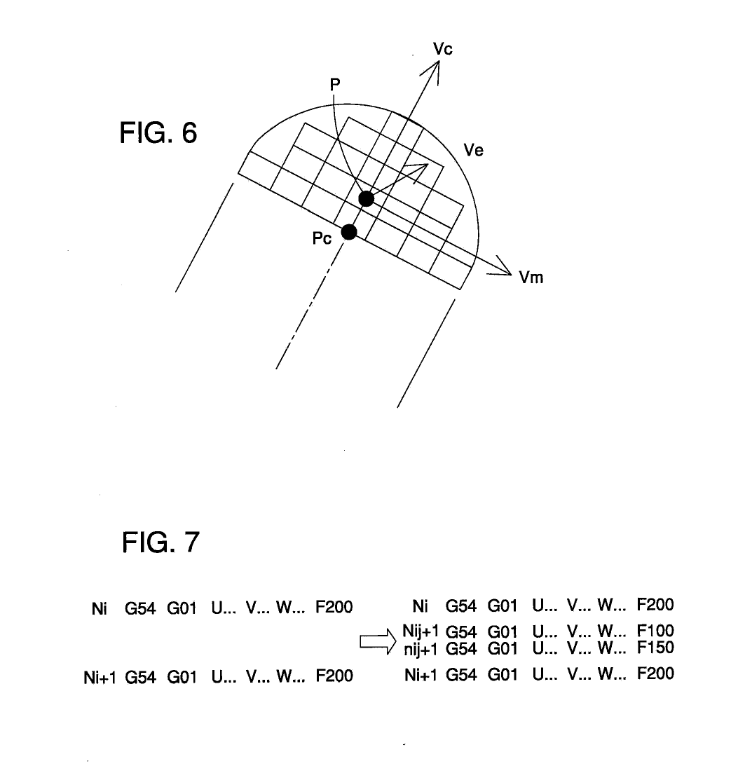

As shown in FIG. 5, when interference determination is performed, for the successive tool position information elements Nᵢ (i=1, 2, 3, 4, . . . m), the spaces between Nᵢ and Nᵢ₋₁ (i=1, 2, 3, 4, . . . m−1) are segmented at predefined intervals, generating the above-described segmented information elements Nᵢⱼ (j=1, 2, 3, 4, . . . n), and calculations are performed for each segmented tool position information element Nᵢⱼ. Since the finite elements 41 subject to determination belong only to regions involved in resection (in the shown example, the cutting edge portion of the ball nose end mill), they can represent the actual resection region 40. As shown in FIG. 6, the extraction of the finite elements 41 of the tool 31 involved in resection is carried out by calculating a dot product of an incision direction vector Vₑ that passes through point P, a tool travel direction vector Vₘ, and a vector V_{c} defined by point P and the central position of the finite elements of the tool, with the proviso that both of the following conditions are satisfied:

$$\begin{matrix}arccos ( \frac{Vc \cdot Ve}{\vert Vc \vert \vert Ve \vert} ) < 90 ( deg ) & ( 2 ) \\ arccos ( \frac{Vm \cdot Ve}{\vert Vm \vert \vert Ve \vert} ) < 90 ( deg ) & ( 3 )\end{matrix}$$

Here, the coordinate value of point P is calculated with reference to the center of the sphere at the distal end of the ball nose end mill in accordance with the following formula, in which the central coordinate value is designated as P_{C}, the spherical radius as r, and the incision depth as d.

$$P = Pc + ( r - d ) \frac{Vc}{\vert Vc \vert} \qquad ( 4 )$$

It should be noted that for the incision direction vector V_{c} and tool travel direction vector Vₘ, the calculations of the above-described formula (2) and (3) are performed by using the method described by the above-described formula (1) to translate their representation in the surgical instrument coordinate system 34, which is used to represent the position of the arm 21 illustrated in FIG. 3, to the voxel coordinate system 32, which is used to represent voxels. These operations and processing define the area involved in the resection performed by the tool 31.

The means 6 for determining the hardness of the bones 23, 24 determines the hardness of the bones 23, 24 in the bone resection region and in the interference region in the above-described tool position information elements In the shown example, the voxels are built from DICOM-formatted CT images; therefore, image brightness values are stored in all the voxel elements. Since these brightness values correspond to CT values, the brightness values are used as CT values. For each voxel element of the voxel region determined in the tool position information elements Nᵢⱼ by the means 5 for determining interference region between the tool 31 and the voxels, the CT values are collected and used to calculate an average value, which serves as a hardness index for the region subject to resection by the tool 31.

Though 8-bit data is used in the present embodiment to illustrate the. CT values, 16-bit data may be used as well. Although the CT values, i.e. the image brightness values, are stored in the DICOM-formatted CT images in the form of 16-bit data, in the present invention, a linear window transformation and a bit transformation are performed and 8-bit data is used for the CT values of all of the bone tissue in order to reduce the volume of information stored in the memory unit 12. Although the reduced information volume will cause a reduction in the transformation accuracy in the case of an inverse transform to CT values, in the bone tissue of the shown example, the CT values possess sufficient width and, therefore, it will not affect the determination of hardness.

In addition, when resection is performed in a composite state that combines bone tissue and soft tissue, the width of the distribution of the CT values increases. In such a case, it is sufficient to set the width and median value used for window transformation such that the CT values of the target tissue are included.

The means 7 for calculating the optimal feed rate of the tool 31 for resecting the bones 23, 24, determines the feed rate by using an average CT value calculated by the means 6 for determining the hardness of the bones 23, 24, as an index of hardness. In general, the CT value is proportional to the density of the biological tissue. For example, it ranges from 0 to 100 in soft tissues, such as liver, etc., and from 50 to 1000 in bone. In this manner, the feed rate is determined by making use of the difference in CT values between soft tissues and hard tissues such as bone, which makes it possible to infer that the tissue is highly dense and hard if the CT value is high and that it is a soft tissue if it is low.

In the bone tissue of the shown example, optimal values are calculated by experimentally determining the relationship between the cutting load and cutting reaction force under resection conditions including parameters such as the feed rate, CT values, and resection conditions (incision depth, tool shape, tool RPM), and these optimal values are stored in the memory unit 12 as specified values. The cutting reaction force is calculated from average CT values corresponding to the tool position information elements Nᵢⱼ obtained by segmentation in the means 6 for determining the hardness of bones 23, 24; and when the cutting reaction force is equal to or higher than a specified value, then an instruction is issued to reduce the currently used feed rate. Conversely, when it becomes smaller than the specified value, an instruction is issued to increase the rate.

The means 8 for out putting optimal feed rates and tool position information elements Nᵢ based on the tool information 30 outputs commands to operate the arm 21 based on the segmented tool position information elements Nᵢⱼ and on the corresponding feed rate increase/decrease instructions. In the present example, as described above, the degrees of freedom of the arm 21 include three (3) translational axes (U, V, W) and thee (3) rotational axes (A, B, C). For this reason, according to conventional practice, the tool information-related operating commands are based on NC (Numerical Control) instructions commonly used in machine tools and are stated relative to a local coordinate system defined by the machine coordinate system (orthogonal UVW axes) represented in the surgical instrument coordinate system 34, as described below.

The tool position information elements Nᵢⱼ are translated to NC instructions and expressed for, for example, the UVWABC axes, which are the working axes, as G54000U2V4W8A20F200. An NC instruction generated by N_{j+I} is outputted subsequent to an NC instruction generated by Nᵢ if there are no feed rate increase/decrease instructions issued by the above-described feed rate calculation means between the tool position information elements Nᵢ and Nᵢ₊₁.

On the other hand, if there is a feed rate increase/decrease instruction issued between the tool position information element Nᵢ and tool position information element. Nᵢ₊₁, e.g. at Nᵢⱼ, then the feed rate of the NC instruction corresponding to the tool position information element Nᵢⱼ₋₁ is increased or decreased. This is due to the fact that increasing or decreasing the feed rate before the tool reaches a hard section, which brings about an increase in the cutting reaction force, or a soft section, which brings about a decrease in the cutting reaction force, minimizes increases and compensates for decreases in the cutting load. If the feed rate has been increased or decreased and no rate instructions are issued at the tool position information element Nᵢⱼ₊₁, an NC instruction is outputted whereby the feed rate is returned to a preset feed rate in a stepwise manner based on subsequent instructions.

The above-described process is carried out with respect to the segmented tool position information elements Nᵢⱼ (i=1, 2, 3, 4, . . . m; j=1, 2, 3, 4, . . . n) and, as illustrated in FIG. 7, a series of NC codes with optimized feed rates is generated.

By way of optimizing the feed rates as described above, it is possible to minimize the vibration of the surgical instrument and the displacement of the biological tissue and to maintain the relative position of the tool 31 at the distal end of the arm 21 with respect to the bones 23, 24 which are subject to resection, thereby allowing for an accurate resection of the bones 23, 24 to be performed. Furthermore, increasing and decreasing the feed rate depending on the hardness of the bones 23, 24 leads to a reduction in processing time while keeping the cutting reaction force at or below a specified value.

In addition, the invention is provided with a real-time feed rate control means based on force feedback from the cutting reaction force. The force acting on the tool 31 is detected by a force sensor 16, such as the one illustrated in FIG. 2. If the cutting reaction force obtained by real-time measurement exceeds the specified value, an instruction indicating that the feed rate is to be reduced in a stepwise manner is outputted directly to the control panel 17 of the arm 21, so that changes in the relative position of the tool 31 and bones 23, 24 subject to resection can be minimized. Conversely, if it is lower than the specified value, an instruction is issued to increase the feed rate.

In general, when biological tissues are resected, it is necessary to perform registration in order to associate the coordinate system used to represent the shape of the biological tissue and processing position information (in this embodiment, the voxel coordinate system 32 illustrated in FIG. 3) with the coordinate system used to represent the biological tissue located in the real space of the operating room (in the present embodiment, the real space coordinate system 33) as described earlier. Errors of 1 mm or less occur during the registration process and these errors are propagated to the tool information 30 calculated by the means 4 of FIG. 1, which calculates a tool path. For this reason, the position of the tool with respect to the position of the biological tissue in real space is shifted by the amount of the registration error, and if this error is large, then the NC-based feed rate increase/decrease instructions may not match the hardness of the biological tissue subject to manipulation, and the tool may encounter hard or soft tissues at a predefined feed rate.

In such a situation, adding a means for real time feed rate control based on force feedback is effective in ensuring accurate tissue resection when there is a large registration error. Although bone is used as an example of biological tissue in the description above, it should be noted that the invention is applicable to other tissues or mixed tissues, and the word “bone” used in the description above should be replaced with a specific biological tissue.

1. A surgical assistance system for operating on biological tissue using a surgical tool attached to an arm of an automatically-controlled surgical instrument, wherein said surgical assistance system comprises
a means for storing and voxelizing medical image data obtained from a biological tissue subject to surgery;
a means for setting an operative location based on a shape of a biological tissue;
a means for calculating a tool path traveled by the surgical tool to perform surgery at the operative location;
a means for determining a region of interference between said surgical tool and voxels;
a means for determining a hardness of the biological tissue in an interference region;
a means for calculating an optimal tool feed rate corresponding to the determined hardness; and
a means for outputting the feed rate obtained by the calculations to said surgical instrument.

2. The surgical assistance system according to claim 1, wherein the hardness of the biological tissue is estimated based on the values of image brightness in medical images, and the feed rate is calculated based on the estimated hardness.

3. The surgical assistance system according to claim 1, wherein a cutting reaction force acting upon said tool is measured in real time, and a feed rate corresponding to the cutting reaction force is calculated based on force feedback.

4. The surgical assistance system according to claim 2, wherein a cutting reaction force acting upon said tool is measured in real time, and a feed rate corresponding to the cutting reaction force is calculated based on force feedback.
